(19) United States
(12) Patent Application Publication
(10) Pub. No.: US 2016/0199350 A1
(43) Pub. Date: Jul. 14, 2016

(54) COMBINATION OF HDAC INHIBITORS WITH THROMBOCYTOPENIA DRUGS

(71) Applicants: Peter Wisdom Atadja, Acton, MA (US); Ricky Wayne Johnstone, Fairfield (AU); Henry Miles Prince, Elsternwick (AU); Mark John Bishton, Melbourne (AU); Simon James Harrison, Melbourne (AU)

(72) Inventors: Peter Wisdom Atadja, Acton, MA (US); Ricky Wayne Johnstone, Fairfield (AU); Henry Miles Prince, Elsternwick (AU); Mark John Bishton, Melbourne (AU); Simon James Harrison, Melbourne (AU)

(21) Appl. No.: 14/954,115

(22) Filed: Nov. 30, 2015

Related U.S. Application Data
(63) Continuation of application No. 13/819,735, filed on Feb. 28, 2013, now abandoned.
(60) Provisional application No. 61/379,238, filed on Sep. 1, 2010.

(51) Int. Cl. A61K 31/4045; A61K 38/22; A61K 31/573; A61K 31/4152; A61K 38/05

(52) U.S. Cl. CPC A61K 31/4045; A61K 31/4152; A61K 38/05; A61K 31/573; A61K 38/22

(57) ABSTRACT

The invention relates to a combination which comprises:
(a) a HDAC inhibitor; and
(b) an anti-thrombocytopenia drug,
   for simultaneous, concurrent, separate or sequential use, especially for use in the treatment of a proliferative diseases. The invention also relates to pharmaceutical compositions or products comprising such a combination, or a method using such a combination

### FIELD OF INVENTION

The invention relates to a combination which comprises:
(a) a histone deacetylase inhibitor (HDACi); and
(b) a drug for the treatment of thrombocytopenia (TCP),
   for simultaneous, concurrent, separate or sequential use, especially for use in the treatment of proliferative diseases, such as cancers, either solid tumor cancer or blood cancer such as leukemia, lymphoma, multiple myeloma (MM), Hodgkin's disease, myelodysplastic syndrome (MDS) or acute myeloblastic leukemia (AML). The invention also relates to pharmaceutical compositions comprising such a combination and to a method of treating thrombocytopenia in a patient receiving a histone deacetylase (HDAC) inhibitor drug. The present invention further also relates to a commercial package or product comprising such a combination.

### BACKGROUND OF INVENTION

Reversible acetylation of histones is a major regulator of gene expression that acts by altering accessibility of transcription factors to DNA. In normal cells, histone deacetylase (HDAC) and histone acetyltrasferase together control the level of acetylation of histones to maintain a balance. Inhibition of HDA results in the accumulation of hyperacetylated histones, which results in a variety of cellular responses. Inhibitors of HDAC (HDACi) have been studied for their therapeutic effects on cancer cells. Recent developments in the field of HDACi research have provided active compounds, both highly efficacious and stable, that are suitable for treating tumors.

Accruing evidence suggests that HDACi are even more efficacious when used in combination with other chemotherapeutic agents. There are both synergistic and additive advantages, both for efficacy and safety. Therapeutic effects of combinations of chemotherapeutic agents with HDACi can result in lower safe dosages ranges of each component in the combination.

### SUMMARY OF INVENTION

This invention relates a pharmaceutical combination of an HDACi with a drug for treating thrombocytopenia (TCP) to treat or delay of progression of a proliferative disease. “Proliferative diseases” include both solid tumor cancers or blood cancers such as leukemia, lymphoma, multiple myeloma, Hodgkin's disease, myelodysplastic syndrome (MDS) or acute myeloblastic leukemia (AML). Preferably, the proliferative disease is multiple myeloma, MDS and/or AML. More preferably, the proliferative disease is multiple myeloma.

HDAC inhibitors are effective when used in combination with an anti-thrombocytopenia drug, especially in patients experiencing HDAC-induced thrombocytopenia. Preferred HDAC inhibitors include panobinostat and vorinostat. Most preferred is panobinostat. The HDAC inhibitor may optionally be used in combination with additional active agents, such as a proteosome inhibitor, an anti-metabolite, and drugs effective for the treatment of multiple myeloma, MDS and/or AML. Preferred drugs to be used in combination with an HDACi include bortezomib and dexamethasone. Preferred anti-metabolites include 5-azacytidine and/or decitabine. Anti-TCP drugs suitable with the present invention comprise thrombopoietin (TPO) mimetics, preferably romiplostim and/or etrombopag.

In a preferred method according to the invention, a use is provided for the combination of (a) N-hydroxy-3-[4-[[[2-(2-methyl-1H-indol-3-yl)-ethyl]-amino]methyl]phenyl]-2E-2-propenamide, or a pharmaceutically acceptable salt thereof in combination with (b) an anti-thrombocytopenia drug in the preparation of a medicament for use as an multiple myeloma drug. The term “use” can also encompass a method treatment, e.g., a method of treating multiple myeloma is provided, which comprises the administration of panobinostat in combination with bortezomib, more preferably in combination with dexamethasone. In yet another preferred embodiment of the present invention, methods for treating MDS and/or AML are provided, which comprise the administration of panobinostat with 5-azacytidine and/or decitabine.

### BRIEF DESCRIPTION OF THE DRAWINGS

FIG. 1 shows lack of effect on platelet apoptosis.

FIG. 2 provides evidence that HDACi-induced thrombocytopenia is likely the result of abberant platelet production.

FIG. 3 provides evidence that TPO-mimetic is effective in ameliorating panobinostat-induced thrombocytopenia.

FIG. 4 provides evidence that TPO-mimetic is effective in ameliorating romidepsin-induced thrombocytopenia.

### DETAILED DESCRIPTION OF INVENTION

The present invention provides a method for delaying the progression of or treatment of a proliferative disease, preferably a blood cancer, more preferably MDS or AML, and most preferably multiple myeloma. The method combines the administration of a drug effective in the treatment of the proliferative disease with a drug effective in the treatment of TCP. Preferably, the method combines the administration of an HDACi with an anti-TCP drug. More preferably, the method combines the administration of an HDACi with an anti-TCP drug in further combination with an additional anti-cancer drug, such as an anti-metabolite. The term “delay of progression” of a disease, as used herein, is in reference to the progression observed or expected in the absence of any treatment.

HDAC inhibitors

One embodiment of the invention provides a method for the delay of progression or treatment of a proliferative disease, preferably multiple myeloma, in a subject in need of such treatment which comprises administering to the subject an effective amount of an HDACi of a hydroxamate of formula (I):
(a) an HDAC of formula (I):
wherein
   R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
   R₂ is selected from H; C₁-C₁₀alkyl, preferably C₁-C₆alkyl, e.g., methyl, ethyl or -CH₂CH₂-OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
   R₃ and R₄ are the same or different and, independently, H; C₁-C₆alkyl; acyl; or acylamino, or
   R₃ and R₄, together with the carbon to which they are bound, represent C═O, C═S or C═NR₈, or
   R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
   R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
   n, n₁, n₂ and n₃ are the same or different and independently selected from 0-6, when n₁ is 1-6, each carbon atom can be optionally and independently substituted with R₃ and/or R₄;
   X and Y are the same or different and independently selected from H; halo; C₁-C₄alkyl, such as CH₃ and CF₃; NO₂; C(O)R₁; OR₉; SR₉; CN; and NR₁₀R₁₁;
   R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
   R₇ is selected from OR₁₅; SR₁₅; S(O)R₁₆; SO₂R₁₇; NR₁₃R₁₄; and NR₁₂SO₂R₆;
   R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
   R₉ is selected from C₁-C₄alkyl, e.g., CH₃ and CF₃; C(O)-alkyl, e.g., C(O)CH₃; and C(O)CF₃;
   R₁₀ and R₁₁ are the same or different and independently selected from H; C₁-C₄alkyl; and —C(O)-alkyl;
   R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
   R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
   R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
   R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
   R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
   R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
   m is an integer selected from 0-6; and
   Z is selected from O; NR₁₃; S; and S(O),
      or a pharmaceutically acceptable salt thereof, in combination with an anti-TCP drug. The combination may further optional comprise the administration of an anti-cancer drug that compromises cell proliferation, such as an anti-metabolite.

Pharmaceutically acceptable salts include, when appropriate, pharmaceutically acceptable base addition salts and acid addition salts, e.g., metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Lactate salt is preferred. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

In one embodiment, dacinostat is the HDACi In a preferred embodiment of the invention, the HDACi is panobinostat N-hydroxy-3-[4-[[[2-(2-methyl-1H-indol-3-yl)-ethyl]-amino]methyl]phenyl]-2E-2-propenamide), or a pharmaceutically acceptable salt thereof, preferably the lactate salt thereof of formula (III) (aka. panobinostat). The present invention also extends to the use of HDAC inhibitors that are not hydroxamates. For example, the invention encompasses the use of Istodax® (romidepsin), a bicyclic tetrapeptide, and Zolinza® (vorinostat), which is suberoylanilide hydroxamic acid.

Anti-TCP Drugs

The present invention contemplates the combination of an anti-thrombocytopenia drug with an HDACi for the treatment of blood cancers. “Anti- thrombocytopenia drugs” encompass thrombopoietin (TPO), including recombinant TPO and pegylated human megakaryocyte growth and development factor (PEG-rhMGDF), and so-called TPO mimetics, which are designed to effectively treat TCP as agonists of the TPO receptor. TPO mimetics include both nonpeptide molecules and peptides. Nplate® (romiplostim, aka AMG 531), for example, is one of the most developed TPO mimetics and is a fusion protein of a TPO receptor-binding peptide and an Fc domain of an IgG1 antibody. Eltrombopag is an exemplary nonpeptide TPO mimetic.

Additional suitable TPO mimetics are described in U.S. Pat. No. 7,160,870, e.g., 3′-{N′-[3-cyclopropyl-1-(3,4-dimethylphenyl)-5-oxo-1,5-dihydropyrazol-4-y-lidene]hydrazino}-2′-hydroxybiphenyl-3-carboxylic acid; [1-(4-fluoro-3-methylphenyl)-3-methyl-5-oxo-1,5-dihydropyrazol-4-ylidene]-hydrazino}-2′-hydroxybiphenyl-3-carboxylic acid; 3′-{N′-[3-methyl-5-oxo-1-(4-trifluoromethylphenyl)-1,5-dihydropyrazol-4-y-lidene]hydrazino}-2′-hydroxybiphenyl-3-carboxylic acid; 3-{N′-[1-(3,4-dimethylphenyl)-3-methyl-5-oxo-1,5-dihydropyrazol-4-ylidene-]hydrazino}-2-hydroxy-3′-tetrazol-5-ylbiphenyl; 3′-{N′-1-(3,4-Dimethylphenyl)-3-methyl-5-oxo-1,5-dihydropyrazol-4-ylidene- Thydrazino}-2′-hydroxybiphenyl-3-carboxylic acid; 3′-{N′-[1-(3-fluoro-4-methylphenyl)-3-methyl-5-oxo-1,5-dihydropyrazol-4-y-lidene]hydrazino}-2′-hydroxybiphenyl-3-carboxylic acid; 3′-{N′-[1-(3,4-dimethylphenyl)-3-ethyl-5-oxo-1,5-dihydropyrazol-4-ylidene-]hydrazino}-2′-hydroxybiphenyl-3-carboxylic acid; and 3-{N′-[1-(3,4-dimethylphenyl)-3-ethyl-5-oxo-1,5-dihydropyrazol-4-ylidene]-hydrazino}-2-hydroxy-3′-tetrazol-5-ylbiphenyl, and preferably 3′-{N′-[1-(3,4-Dimethylphenyl)-3-methyl-5-oxo-1,5-dihydropyrazol-4-yliden-e]hydrazino}-2′-hydroxybiphenyl-3-carboxylic acid, and a pharmaceutically acceptable salt, a hydrate, a solvate, and an ester, thereof.

As noted herein, the combination of HDACi and anti-TCP medicament may be further combined with an additional medicament, preferably an anti-cancer drug. More preferably, the anti-cancer drug is one effective in the treatment of blood cancers such as multiple myeloma, MDS and/or AML. Such drugs can include anti-metabolites, such as Vidaza® (5-azacytidine) and/or Dacogen® (decitabine) and proteosome inhibitors, such as Velcade® (bortezomib).

Thrombocytopenia is any disorder in which there is an abnormally low amount of platelets, such as having below 50,000 platelets per microliter or being in the lower 2.5 percentile of the normal (average or median) platelet count for a particular human population. TCP has many causes, but the etiology underlying HDACi-induced TCP has not been elucidated. The present inventors have discovered that HDACi-induced TCP is due to decreased platelet production or platelet release from megakaryocytes, and not myelosuppression, myeloablation or reduced platelet lifespan (e.g., apoptosis) as was commonly believed in the art. Hence, without being held to or bound by theory, the present invention provides for the treatment of TCP observed in HDACi treatment with anti-TCP drugs that specifically address platelet production. In this way, side-effects observed with the administration of most conventional anti-TCP medications may be curbed or avoided.

In a preferred embodiment of the invention, the drug for treating TCP is eltrombopag or romiplostim. In a preferred embodiment, eltrombopag or romiplostim is used in combination with an HDACi, such as panobinostat, or a pharmaceutically acceptable salt thereof.

Further the invention provides the use of a HDAC inhibitor, or pharmaceutically acceptable salt or prodrug ester thereof, for the preparation of a medicament for use in combination with an anti-thrombocytopenia drug in the treatment of a proliferative disease.

“Combination” refers to administration of an amount of HDAC inhibitor in combination with administration of an amount of an anti-thrombocytopenia drug such that there is a synergistic effect, which would not be obtained if an HDAC inhibitor were administered without separate, simultaneous or sequential administration of the anti-thrombocytopenia drug. Administration of an anti-thrombocytopenia drug can be continuous, sequential or sporadic. Accordingly, “medicament”, as used herein, should not be limited to a single formulation comprising the inventive combination, but open to a regimen or treatment comprising the administration of active agents of the inventive combination in distinct dosage forms.

Preferably, combination refers to administration of an amount of HDAC inhibitor in combination with administration of an amount of an anti-thrombocytopenia drug such that there is a synergistic anti-proliferative effect and/or a clonogenic cell killing effect that would not be obtained if:
a) The HDAC is administered without prior, simultaneous or subsequent administration of an anti-TCP drug. Wherein administration can be continuous, sequential or sporadic;
b) There is administration of an anti-thrombocytopenia drug without the prior, simultaneous or subsequent administration of an HDAC inhibitor, wherein administration can be continuous, sequential or sporadic.

Hence, a combination which comprises:
(a) an HDAC inhibitor, which may be present in free form or in the form of a pharmaceutically acceptable salt and optionally at least one pharmaceutically acceptable carrier; and
(b) an anti- thrombocytopenia drug, will be referred to hereinafter as a combination of the invention.

Synergy may be observed between an HDACi and anti-TCP medication such that a lower dose of the anti-TCP may be effective in treating TCP than would otherwise be required in the absence of HDACi co-treatment. For example, the recommended dose of romiplostim ranges from 1 mg/kg to 10 mg/kg. Accordingly, the dosage of an anti-thrombocytopenia drug and an HDAC inhibitor in relation to each other is preferably in a ratio that is synergistic. In one embodiment of the invention HDACi co-treatment can reduce the effective dosage of TCP by 5% or 10%, preferably 15% or 20%, most preferably 30% to 40%.

Conversely, synergy may also be observed between an HDACi and anti-TCP medication such that a lower dose of the HDACi may be effective in treating cancer than would otherwise be required in the absence of TCP co-treatment. For example, the recommended doses of panobinostat can be expressed as 20-40 mg three times a week or from 15 mg/kg to 20 mg/kg. In a preferred embodiment of the invention, anti-TCP co-treatment can reduce the effective dosage of panobinostat by 5% or 10%, preferably 15% or 20%, most preferably 30% to 40%. For example, co-administration of romiplostim could reduce the effective dose of panobinostat from 20 mg/kg to 15 mg/kg.

Synergy may also be observed between an HDACi and an additional drug when used the HDACi is used in combination with an anti-TCP medication. For example, synergy may be observed between panobinostat and dexamethasone such that a lower dose of the HDACi may be may be effective in treating multiple myeloma than would otherwise be required in the absence of dexamethasone co-treatment. In one embodiment of the invention a further combination with an additional drug such as dexamethasone can reduce the effective dosage of HDACi by 5% or 10%, preferably 15% or 20%, most preferably 30% to 40%.

In the combination of the invention, HDAC inhibitor and pharmaceutically acceptable salts and prodrug derivatives are preferably used in the form of pharmaceutical preparations that contain the relevant therapeutically effective amount of active ingredient optionally together with or in admixture with inorganic or organic, solid or liquid, pharmaceutically acceptable carriers which are suitable for administration. Preferably, the HDAC pharmaceutical compositions are adapted to oral administration.

The HDACi and anti-TCP pharmaceutical compositions, individually or in combination, may be, e.g., compositions for enteral, such as oral, rectal, aerosol inhalation or nasal administration, compositions for parenteral, such as intravenous or subcutaneous administration, or compositions for transdermal administration (e.g., passive or iontophoretic), or compositions for topical administration.

Preparation

The pharmaceutical compositions according to the invention can be prepared in any manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

The novel pharmaceutical composition contain, e.g., from about 10% to about 100%, preferably from about 20% to about 60%, of the active ingredients. Pharmaceutical preparations for the combination therapy for enteral or parenteral administration are, e.g., those in unit dosage forms, such as sugar-coated tablets, tablets, capsules or suppositories, and furthermore ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, e.g., water, glycols, oils, alcohols, flavouring agents, preservatives, colouring agents; or carriers such as starches, sugars, microcrystalline cellulose, diluents, granulating agents, lubricants, binders, disintegrating agents and the like in the case of oral solid preparations, such as, e.g., powders, capsules and tablets, with the solid oral preparations being preferred over the liquid preparations. Because of their ease of administration, tablets and capsules represent the most advantageous oral dosage unit form in which case solid pharmaceutical carriers are obviously employed.

A therapeutically effective amount of each combination partner of the combination of the invention may be administered simultaneously or sequentially and in any order, and the components may be administered separately or as a fixed combination. For example, the method of delay of progression or treatment of a proliferative disease according to the invention may comprise:
(i) administration of the first combination partner; and
(ii) administration of the second combination partner,
   wherein administration of a combination partner may be simultaneous or sequential in any order, in jointly therapeutically effective amounts, preferably in synergistically effective amounts, e.g., in daily or weekly dosages corresponding to the amounts described herein. The individual combination partners of the combination of the invention can be administered separately at different times during the course of therapy or concurrently. In a preferred embodiment, the anti-thrombocytopenia drug is given as a pre-treatment, i.e. before the treatment with an HDACi is started; the anti-thrombocytopenia drug alone is administered to the patient for a defined period of time.

Administration

Furthermore, the term “administering” also encompasses the use of a pro-drug of an HDAC inhibitor or an anti-TCP drug that converts in vivo to the combination partner as such. The instant invention is therefore to be understood as embracing all such regimes of simultaneous or alternating treatment and the term “administering” is to be interpreted accordingly.

If the warm-blooded animal is a human, the dosage of a compound of formula (I) is preferably an appropriate dose in the range from 100-1,500 mg daily, e.g., 200-1,000 mg/day, such as 200, 400, 500, 600, 800, 900 or 1,000 mg/day, administered in one or two doses daily. Appropriate dosages and the frequency of administration of the death receptor ligand will depend on such factors, as the nature and severity of the indication being treated, the desired response, the condition of the patient and so forth.

The particular mode of administration and the dosage of an HDAC inhibitor may be selected by the attending physician taking into account the particulars of the patient, especially age, weight, life style, activity level, etc. Similarly, the dosage of an HDAC inhibitor may depend on various factors, such as effectiveness and duration of action of the active ingredient, mode of administration, effectiveness and duration of action of the ionizing radiation and/or sex, age, weight and individual condition of the subject to be treated.

The dosage of ionizing radiation may depend on various factors, such as effectiveness and duration of action of the ionizing radiation, mode of administration, location of administration, effectiveness and duration of action of the HDAC inhibitor and/or sex, age, weight and individual condition of the subject to be treated. The dosage of ionizing radiation is generally defined in terms of radiation absorbed dose, time and fraction, and must be carefully defined by the attending physician.

In one preferred embodiment of the invention the combination comprises an anti-thromboxytopenia drug, such as eltrombopag and N-hydroxy-3-[4-[[[2-(2-methyl-1H-indol-3-yl)-ethyl]-amino]methyl]phenyl]-2E-2-propenamide, of formula (III) above or a pharmaceutically acceptable salt thereof. In another preferred embodiment of the invention the combination comprises an anti-thromboxytopenia drug, such as romiplostim and N-hydroxy-3-[4-[[[2-(2-methyl-1H-indol-3-yl)-ethyl]-amino]methyl]phenyl]-2E-2-propenamide, of formula (III) above or a pharmaceutically acceptable salt thereof. In both embodiments, the combination may further include the preferential administration of a drug that inhibits cell proliferation, such as bortezomib.

Moreover, the present invention relates to a method of treating a warm-blooded animal having a proliferative disease comprising administering to a human patient in a way that is jointly therapeutically effective against a proliferative disease and in which the combination partners can also be present in the form of their pharmaceutically acceptable salts. The combination can work in a way that inhibits thrombocytopenia or its related symptoms

Furthermore, the present invention pertains to the use of a combination of the invention for the delay of progression or treatment of a proliferative disease and for the preparation of a medicament for the delay of progression or treatment of a proliferative disease.

### EXAMPLES

The following examples are merely illustrative and not meant to limit the scope of the present invention in any manner:

### Example 1

### Combination of Compound of Formula III with Eltrombopag in Hodgkin's Lymphoma Patient

A patient having Hodgkin's lymphoma is given the recommend dosage of compound III. The patient's platelet count begins to lower. Patient is then given eltrombopag and the patient's platelet blood count begins to rise to an acceptable level.

### Example 2

### Combination of Compound of Formula III with Eltrombopag in Hodgkin's Lymphoma Patient

A patient having Hodgkin's lymphoma is tested for thrombocytopenia. Patient is found to have a low blood cell count or a biomarker for thrombocytopenia. Patient is put on a regimen of compound III together with eltrombopag. Patient does not develop thrombocytopenia.

### Example 3

### Combination of Compound of Formula III with Eltrombopag in Multiple Myeloma Patient

A patient having multiple myeloma patient is given the recommend dosage of compound III. The patient's platelet count begins to lower. Patient is then given eltrombopag and the patient's platelet blood count begins to rise to an acceptable level.

### Example 4

### Combination of Compound of Formula III with Eltrombopag in Multiple Myeloma Patient

A patient having Hodgkin's lymphoma is tested for thrombocytopenia. Patient is found to have a low blood cell count or a biomarker for thrombocytopenia. Patient is put on a regimen of compound III together with eltrombopag. Patient does not develop thrombocytopenia.

### Example 5

### Platelet Clearance Analysis and Reticulated Platelet Staining

Mice were twice injected intravenously (IV), one hour apart, with 600 μg NHS-biotin in phospho-buffered saline (PBS) and 10% dimethyl sulphoxide (DMSO). At various time points peripheral blood was isolated from the tail, and 1 μl blood was washed twice in PBS and 10 mM EDTA. Platelets were stained with phycoerythrin-conjugated rat anti-CD41 and allophycocyanin-conjugated streptavidin (APC-A) for 30 minutes on ice. Samples were washed again and incubated with 0.125 μg/ml thiazole orange, which has increased uptake in high RNA-containing platelets, staining younger, reticulated platelet fraction, for 90 minutes. Flow cytometry was then performed on an LSR flow cytometer. By plotting the number of biotinylated platelets against time, an estimate of the life span was obtained by linear extrapolation, while the number of thiazole orange-positive, non-biotinylated platelets provided an estimate of new platelet production.

### Example 6

### HDACi-induced Thrombocytopenia is Not Attributable to Direct Platelet Apoptosis

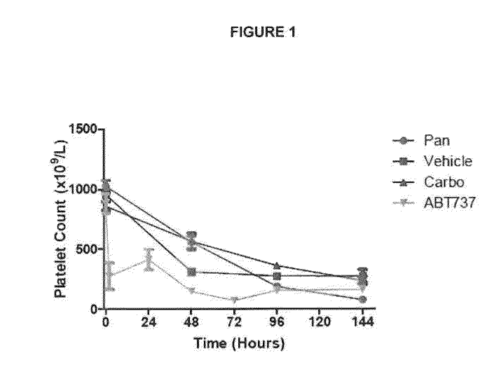

To further confirm that direct platelet apoptosis was not the cause of HDACi-induced TCP, murine platelet lifespan was assessed in mice treated with HDACi by injecting mice with IV NHS-biotin. The mice were then treated for seven days with either 10 mg/kg panobinostat IP or 1 mg/kg romidepsin IP daily or vehicle, and the number of biotinylated platelets in peripheral blood was determined. The decrease in labelled platelets over time remained similar to vehicle-treated mice compared to panobinostat- or romidepsin-treated mice. In contrast, the number of biotinylated platelets rapidly reduced following treatment of mice with ABT-737 with a 50% reduction in platelet number seen two hours following administration of the compound. ABT-737 is a BH3 mimetic that inhibits Bcl-xL, which causes direct platelet apoptosis. Carboplatin, a chemotherapeutic agent capable of inducing TCP by megakaryocyte ablation did not affect platelet life span (FIG. 1). Megakaryocytes are the haematopoietic cells responsible for the production of platelets which reside in the bone marrow.

### Example 7

### HDACi-induced Thrombocytopenia is Due to a Reduction in Platelet Production

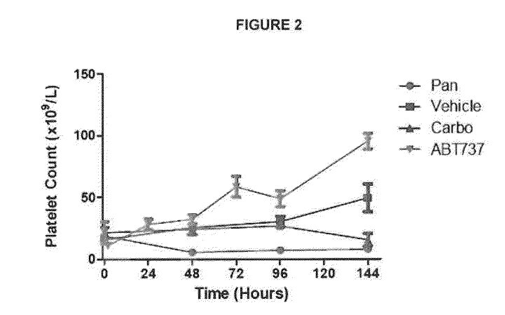

To assess platelet production, C57BL/6 mice were treated with panobinostat, carboplatin and ABT-737, and the RNA-containing platelet fraction (i.e. new platelets) was determined by staining with thiazole orange (FIG. 2). The number of new, reticulated platelets in vehicle-treated mice remained relatively constant over the 6 day time span of the experiment. In contrast, treatment with panobinostat resulted in a dramatic decrease in the absolute number of reticulated platelets. The platelet count remained well below baseline levels throughout the course of the experiment. ABT-737 caused a substantial increase in production of new platelets as a compensatory response to the rapid induction of platelet apoptosis. Treatment of mice with carboplatin did not significantly affect new platelet production for the first 4 days of treatment, however, absolute reticulated platelet numbers were significantly reduced 6 days after the commencement of treatment. Taken together these data indicate that ABT-737, carboplatin and panobinostat mediate TCP via different mechanisms. We hypothezised that HDACiinduced TCP was due to inadequate platelet production or poor platelet release from the megakaryocyte, rather than myeloablation or direct platelet apoptosis as seen with carboplatin and ABT-737, respectively.

### Example 7

### Combination of Panobinostat with Romiplostim

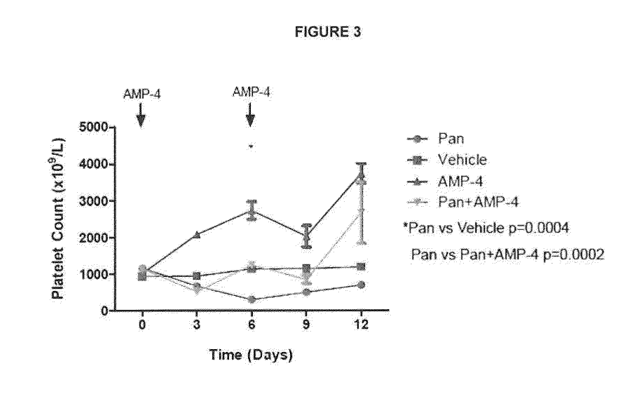

Wild-type C57BL/6 mice were treated with 10 mg/kg IP daily panobinostat, which caused sustained TCP over a 12-day period consistent with previous results, or 20 μg/kg at days three and six of the TPO-mimetic AMP-4. To determine whether administration of a TPO-mimetic could ameliorate TCP, some of the panobinostat-treated mice were subjected to co-treatment with 20 μg/kg AMP-4 at day 0 and day 6 (FIG. 3). AMP-4 is another TPO mimetic that has an identical binding peptide as romiplostim but has a murine Fc receptor.

### Example 8

### Combination of Romidepsin with Romiplostim

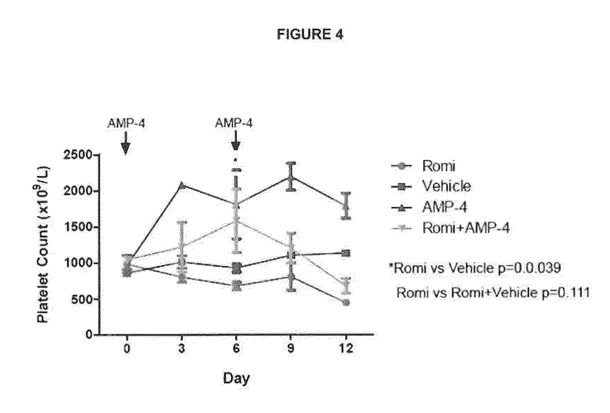

Similarly, wild-type C57BL/6 mice were treated with 10 mg/kg IP daily romidepsin, which caused sustained TCP over a 12-day period consistent with previous results, or 20 μg/kg at days three and six of the TPO-mimetic AMP-4. To determine whether administration of a TPO-mimetic could ameliorate TCP, some of the romidepsin-treated mice were subjected to co-treatment with 20 μg/kg AMP-4 at day 0 and day 6 (FIG. 4). AMP-4 is another TPO mimetic that has an identical binding peptide as romiplostim but has a murine Fc receptor.

These data provide the first evidence of a treatment regimen that overcomes HDACi-induced TCP.

What is claimed is:

1. The combination of (a) N-hydroxy-3-[4-[[[2-(2-methyl-1H-indol-3-yl)-ethyl]-amino]methyl]phenyI]-2E-2-propenamide, or a pharmaceutically acceptable salt thereof in combination with (b) an anti-thrombocytopenia drug.

2. The combination according to claim 1, wherein the anti- thrombocytopenia drug is an TPO mimetic.

3. The combination according to claim 2, wherein the anti-thrombocytopenia drug is eltrombopag, romiplostim, or both.

4. The combination according to any of claim 3, wherein the combination further comprises an additional drug effective for the treatment of multiple myeloma.

5. The combination according to claim 4, wherein the further drug is a proteosome inhibitor.

6. The combination according to claim 5, wherein the proteosome inhibitor is bortezomib.

7. The combination according to claim 6, wherein the combination further comprises dexamethasone.

8. The combination of:
(a) an HDACi inhibitor selected from the group consisting of panobinostat, romidepsin, vorinostat, and combinations thereof;
(b) an anti-TCP drug selected from the group consisting of eltrombopag, romiplostim, or both; and, optionally,
(c) an anti-metabolite selected from the group consisting of 5-azacytidine, decitabine, or both.

9. A method of treating multiple myeloma in a patient in need therefore, comprising administering to the patient the combination of claim 1.

10. A method of treating MDS or AML in a patient in need therefore, comprising administering to the patient the combination of claim 8.

11. The method of claim 10, wherein the patient is in need of treatment of MDS.

12. The method of claim 10, wherein the patient is in need of treatment of AML.
